(19) United States
(12) Patent Application Publication
(10) Pub. No.: US 2008/0153174 A1
(43) Pub. Date: Jun. 26, 2008

(54) CATALYTIC ALLOY HYDROGEN SENSOR APPARATUS AND PROCESS

(76) Inventors: Douglas B. Galloway, Mount Prospect, IL (US); Randall E. Holt, Elgin, IL (US); Patrick J. Bullen, Elmhurst, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/613,264

(22) Filed: Dec. 20, 2006

(51) Int. Cl. G01N 33/00

(52) U.S. Cl. 436/144

(57) ABSTRACT

A process for sensing hydrogen in a refinery process stream or a chemical process stream has been developed. The process comprises flowing at least a portion of the refinery process stream or the chemical process stream through a conduit which is part of a main support and through a series of flow components which are attached to the main support and in fluid communication with the conduit. The flow components comprise, a needle valve, a pressure indicator, a catalytic alloy hydrogen sensor, and a back pressure regulator. The catalytic alloy hydrogen sensor may be a palladium-nickel catalytic alloy hydrogen sensor. The process continues with generating a signal indicating the amount of hydrogen in the stream using the catalytic alloy hydrogen sensor and communicating the signal to a display device or a computer processor. The process may further include adjusting an operating parameter based upon the signal indicating the amount of hydrogen in the stream, or based upon a calculated mole percent hydrogen. The process of the invention may be used for controlling a refinery or chemical process.

### FIELD OF THE INVENTION

The present invention is related to hydrogen sensors, and more particularly, to an assembly for a modular hydrogen sensor system using a catalytic alloy hydrogen sensor.

### BACKGROUND OF THE INVENTION

Chemical sensing equipment has long been helpful in monitoring processes. Hydrogen sensors in particular have been employed in a variety of applications. Improvements in hydrogen sensors have resulted in sensors that are capable of detecting a wide range of hydrogen concentrations with reproducible signals. They respond rapidly and reversibly to changes in hydrogen concentration and exhibit resistance to poisoning.

One particular class of hydrogen sensors began in the early 1990s when Sandia National Laboratory developed a single-chip hydrogen sensor that utilized Palladium-Nickel (PdNi) catalytic alloy as hydrogen gas sensors. The PdNi catalytic alloy was deposited on a metal-oxide semiconductor (CMOS), see U.S. Pat. No. 5,279,795 which is incorporated by reference herein. One of the key benefits of the sensor described in the '795 patent is its ability to detect a dynamic range of hydrogen concentrations over at least six orders of magnitude. Prior solid state sensor solutions to the problem of detecting hydrogen concentrations had been generally limited to detecting low concentrations of hydrogen. These solutions include such technologies as metal-insulator-semiconductor (MIS) or metal-oxide-semiconductor (MOS) capacitors and field-effect-transistors (FET), as well as palladium-gated diodes. The '795 sensor also provided reliable performance over a large temperature range, about 100° C. to about 140° C. and was dependable for operating in diverse environments such as vacuum, non-oxygen ambient, hostile vibrations, and radiation conditions.

In general, the PdNi catalytic alloy has proven to be successful in many applications, but different alloys may be used to sense hydrogen as well. Examples include, nickel with either catalytic metals such as platinum, rhodium as well as alloys of palladium and copper, palladium and platinum, and platinum and chromium are also effective.

The hydrogen sensor described in the '795 patent was a notable advance in hydroponic-sensing technology. It was, however, primarily limited to an experimental laboratory environment due to the difficulties encountered in manufacturing such a sensor. Difficulties in producing such semiconductor devices due to the specialized materials were believed to result in low device production yields. An economically feasible commercial hydrogen sensor is difficult to obtain if yields are under an acceptable level.

Several techniques were developed to improve device yields in an attempt to manufacture a commercializable single-chip hydrogen sensor. Two of these techniques are described respectively in U.S. Pat. No. 6,450,007 titled “Robust Single-Chip Hydrogen Sensor,” U.S. Pat. No. 6,730,270 titled “Manufacturable Single-Chip Hydrogen Sensor,” and U.S. Pat. No. 6,634,213 titled “Permeable Protective Coating for a Single-Chip Hydrogen Sensor” all of which are incorporated by reference herein. Today, several different types of hydrogen sensors using the PdNi catalytic alloy originally invented at Sandia National Laboratory are commercially available.

From a general perspective, these hydrogen sensors operate through changes in the resistance or conductance in the catalytic alloy upon the adsorption of hydrogen. When the alloy is exposed to an environment containing hydrogen, the palladium metal component of the catalytic alloy catalyzes the reaction of molecular hydrogen, H₂, into atomic hydrogen, 2 H. The atomic hydrogen then moves into the lattice of the PdNi alloy film. An equilibrium hydrogen density is reached in the alloy which is proportional to the concentration of hydrogen in the gaseous environment of the alloy. Hydrogen absorbed into the PdNi alloy lattice changes the charge density in the alloy lattice which results in an electrical change in the alloy, not a chemical change in the alloy. By this mechanism the device senses H₂ partial pressure.

The sensors have exhibited a rapid response time to changes in hydrogen in the environment of the sensor. The '795 patent demonstrated the rapid response of the sensor by tracking the response time to a cyclic exposure of a gas containing 1% hydrogen followed by a purge of the hydrogen. This experiment also demonstrated that the sensor response was reversible. When hydrogen was removed from the environment, the sensor tracked the loss of hydrogen as rapidly as it had detected the presence of hydrogen.

Finally, because the mechanism for detection is an electrical change in the catalytic alloy, the sensor experiences no interference from hydrocarbons. This feature is especially important when considering possible applications for the sensors. Not all hydrogen sensors in the art will function adequately in every application where hydrogen is to be monitored or measured. Sensors employing differing technologies have unique limitations. For example, one type of sensor may experience significant interferences from a component found in an environment whereas a second type of sensor may function successfully in the same environment. Having a hydrogen sensor free of interferences from hydrocarbons opens a host of applications in fields where hydrocarbons are common such as refining, and chemicals including petrochemicals and specialty chemicals. Other applications may include hydrogen purification operations, pressure swing adsorption processes and controlling or monitoring waste streams. Furthermore, the hydrogen sensor used in the present invention may operate at higher temperatures than other sensors and provides output faster than other sensors, and therefore a wider scope of applications may employ the sensor.

Previously, the potential applications of the catalytic alloy hydrogen sensor that have been considered include: sensing hydrogen buildups in lead acid storage cells found in most vehicles; detecting hydrogen leaks during ammonia or methanol manufacturing; desulfurization of petroleum products; petrochemical applications where high pressure hydrogen is used; detecting impending transformer failure in electric power plants; monitoring hydrogen buildup in radioactive waste tanks and in plutonium reprocessing; and detecting hydrogen leaks during space shuttle launches and other National Aeronautics and Space Administration (NASA) operations. Since the PdNi catalytic alloy was invented, it has been used in different applications and has been modified for ease of manufacturing and ease of use.

However, there remains a need to move beyond merely monitoring hydrogen levels, and instead actually controlling refining and chemical processes based on the concentration of hydrogen at one or more locations of the process. The process locations where hydrogen is measured typically involve a hydrocarbon environment. The catalytic alloy hydrogen sensor may be used to measure the concentration of hydrogen at one or more locations of the process and the value determined used in a feedback loop to control the process by comparing the measured value to a predetermined range of values and if necessary making one or more adjustments to one or more operating parameters. Often, the control process takes place over time with periodic measurements of the hydrogen concentration, comparison to predetermined values, and adjustments to operating parameters.

There also remains a need to have the hydrogen sensor in a format readily adaptable for use in monitoring hydrogen in various petroleum refining and chemical processes. Once integrated into a format that is adaptable for use in refining processes, the range of applications for the sensors is greatly increased. The sensors are no longer merely for monitoring for leaks of hydrogen, but may be used to monitor and control the refining and chemical processes themselves. The format of the assembly containing the sensor should be modular, adaptable, reliable, and easy to use.

As one embodiment of the invention, the sensor is integrated into an appropriate assembly which can be used in a feedback loop to control one or more operating parameters of a refinery or chemical process. The assembly may be supported by a main support and have a needle valve, a pressure indicator, the catalytic alloy sensor, and a back pressure regulator. Optional additional components are a filter, a check valve, and a thermocouple. Apparatus for calibrating the sensor may be associated with, or part of, the assembly as well. In one embodiment, the sensor may additionally contain integrated temperature control. In another embodiment, the sensor may contain integrated pressure indicator. In yet another embodiment, the sensor may contain a processor to calculate the mole percent hydrogen in a stream using the pressure measurement and the hydrogen measurement from the sensor.

The assembly may be used to control refining or chemical processes that produce or consume hydrogen, use hydrogen as a desorbent, or that use hydrogen as a diluent. Examples of processes include cracking, hydrocracking, aromatic alkylation, isoparaffin alkylation, isomerization, polymerization, reforming, dewaxing, hydrogenation, dehydrogenation, transalkylation, dealkylation, hydration, dehydration, hydrotreating, hydrodenitrogenation, hydrodesulfurization, methanation, ring opening, syngas shift, and hydrogen purification. A specific example is one where the assembly is used to control the processes to establish the most optimum time intervals for changing the adsorption or desorption cycles in an isomerization process which uses an adsorbent for separating hydrocarbons. The refinery or chemical units that may be controlled using the present invention include examples such as a reactor, a fractionation unit, an adsorptive separation unit, an extraction unit, a reaction with distillation unit, a vapor liquid contacting device, and a hydrogen purification unit.

### SUMMARY OF THE INVENTION

A process for sensing hydrogen in a refinery process stream or a chemical process stream has been developed. The process comprises flowing at least a portion of the refinery process stream or the chemical process stream through a conduit which is part of a main support and through a series of flow components which are attached to the main support and in fluid communication with the conduit. The flow components comprise, a needle valve, a pressure indicator, a catalytic alloy hydrogen sensor, and a back pressure regulator. The catalytic alloy hydrogen sensor may be a palladium-nickel catalytic alloy hydrogen sensor. The process continues with generating a signal indicating the amount of hydrogen in the stream using the catalytic alloy hydrogen sensor and communicating the signal to a display device or a computer processor. Optionally, additional flow components may be used such as a filter, a check valve, and a thermocouple. The process may include generating a signal indicating the pressure in the stream using the pressure indicator, communicating the signal to a computer processor and calculating, in the computer processor, the mole percent of hydrogen in the stream from the signal indicating the pressure from the pressure indicator and the signal indicating the amount of hydrogen from the catalytic alloy hydrogen sensor. The process may further include adjusting an operating parameter based upon the signal indicating the amount of hydrogen in the stream, or based upon the calculated mole percent hydrogen. The process of the invention may be used for controlling a refinery or chemical process.

### BRIEF DESCRIPTION OF THE DRAWINGS

FIG. 1 shows an assembly for sampling a portion of a refinery or chemical process stream wherein the assembly contains an integrated catalytic alloy hydrogen sensor.

FIG. 2 is a schematic representation of a generic total isomerization process modified and operated in accordance with the process of this invention. The drawing has been simplified by the deletion of a large number of pieces of apparatus customarily employed on processes of this nature which are not specifically required to illustrate the performance of the present invention.

FIG. 3 a schematic representation of the adsorptive separation portion of a generic total isomerization process modified and operated in accordance with the process of this invention. The drawing has been simplified by the deletion of a large number of pieces of apparatus customarily employed on processes of this nature which are not specifically required to illustrate the performance of the present invention.

FIGS. 4A and 4B are plots of data generated using the present invention.

FIGS. 5A and 5B are plots of data generated using the present invention.

FIG. 6 is a plot of data showing the effectiveness of the present invention on a total isomerization process.

### DETAILED DESCRIPTION OF THE INVENTION

A catalytic alloy hydrogen sensor such as those described in U.S. Pat. No. 5,279,795 is integrated into an assembly which is readily adaptable for use in refining and chemical processes. The catalytic alloy hydrogen sensor may be improved from that described in U.S. Pat. No. 5,279,795. For example, the sensor may have integrated temperature control, or integrated pressure indicator. One example of a suitable assembly is provided by the New Sampling/Sensor Initiative (NeSSI). This initiative has developed modular sampling systems with simple building block-like assembly. The sampling systems are easy to reconfigure and install. The flow components of the system are standardized for mix-and-match compatibilities between vendors and the electrical and communication features are plug-and-play. The standard mechanical interface for all components is the rail or platform, upon which is placed flow controllers, sensors, and other equipment. A standard electrical interface with the rail provides connectivity with a computer and other devices. Examples of flow controllers include metering valve, regulator, relief valve, adapter, toggle, check valve, needle valve, non-spill quick disconnect, in line and bypass filters, and manual diaphragms. Examples of sensors incorporated into these sampling systems include dielectric sensors, Raman sensors, and oxygen sensors. Pressure and temperature transducers may also be included on the rail. Other sampling systems may be used, such as the more traditional method of slip stream sampling and routing to the sensor, or probes or collecting individual aliquots of sample for off-line analysis or at-line analysis, or finally, directly from the processes line without the use of the slip stream.

Further, it in envisioned that the present invention may be employed as a portable unit which can be attached to a sampling manifold. In this way, one device may be transported from refinery to refinery or plant to plant, or from one location to another within a refinery or plant, resulting in cost savings. The sampling manifold(s) would be used to route at least a portion of the stream of interest to the catalytic alloy hydrogen sensor assembly.

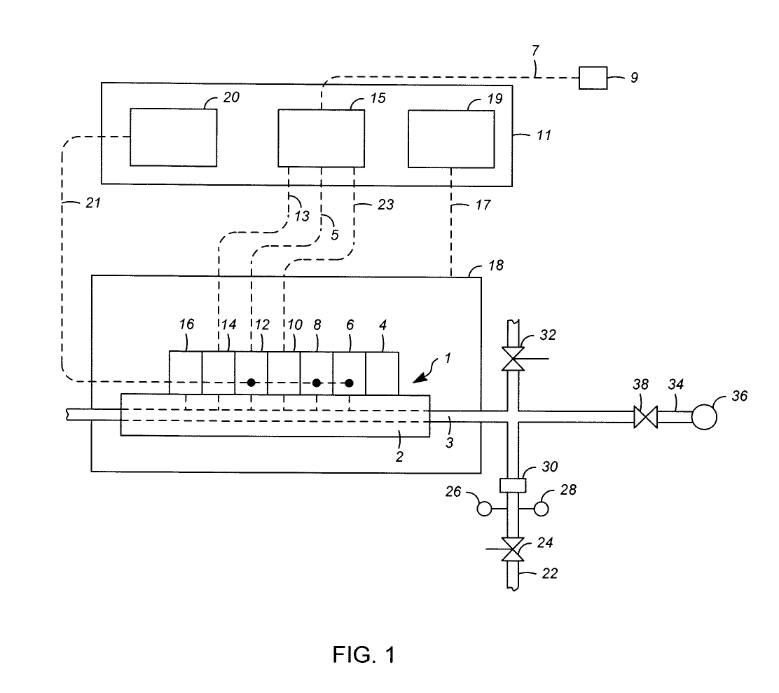

FIG. 1 shows a modular assembly which has an integrated catalytic alloy hydrogen sensor. A main support 2 having a fluid conduit 3 is attached to several flow components and sensors which collectively form assembly 1. The flow components are in fluid communication with the fluid conduit 3. Attached to main support 2 is needle valve 4, filter 6, check valve 8, pressure indicator 10, thermocouple 12, catalytic alloy hydrogen sensor 14, and back pressure regulator 16. Note that filter 6, check valve 8, and thermocouple 12 are optional flow components. The catalytic alloy hydrogen sensor 14 is an integrated part of assembly 1. Valve 4 and back pressure regulator 16 are used to control the flow rate and pressure of the material passed through catalytic alloy hydrogen sensor 14. Filter 6 is used to remove any particulate matter that may be present and prevent fouling of catalytic alloy hydrogen sensor 14. Pressure indicator 10 provides readings of the pressure and thermocouple 12 provides readings of the temperature. Check valve 8 provides that backward flow does not occur in the assembly. As the flow is passed though catalytic alloy hydrogen sensor 14 the sensor generates a signal which may be monitored and tracked for indications of relative concentrations of hydrogen or trending of the concentration of hydrogen. But since the sensor signal is an indication of the hydrogen partial pressure, the pressure as indicated by the pressure indicator is used along with the hydrogen partial pressure signal from the hydrogen sensor to calculate the mole percent of hydrogen in the material being measured. The calculations may be performed by the computer. For ease of explanation, the discussion below refers to the signal from the hydrogen sensor in general and it is understood that the signal may be the hydrogen partial pressure from the hydrogen sensor, or may be a quantitative determination such as the mole percent hydrogen calculated through applying the measured pressure of the sample to the hydrogen partial pressure. Other quantitative concentrations of hydrogen such as mass percent hydrogen or volume percent hydrogen may be calculated. For ease of understanding, mole percent hydrogen will be used as the example to describe the invention. The signal from catalytic alloy hydrogen sensor 14 is conducted via electrical connection 13 to computer processor 15. The signal indicating a pressure measurement is conducted from pressure indicator 10 to computer processor 15 via electrical connection 23, which is optional. The signal indicating a temperature measurement is conducted from thermocouple 12 to computer processor 15 via electrical connection 5, which is optional.

The assembly 1 is contained within optional chamber 18 to keep the process material at the proper temperature. The temperature of the chamber may be adjustable for different applications or different points in time of the same application. For example, the chamber may be used to maintain the fluid in the vapor phase. Therefore, chamber 18 may be equipped with temperature controller 19 which is connected to chamber 18 via electrical connection 17. Various components of the assembly may need power to function, and so components may be connected to one or more power sources 20. In FIG. 1 pressure transducer 6, actuator 8, and thermocouple 12 are all connected to power source 20 via electrical connection 21. Associated with assembly 1 is electrical box 11 which houses low voltage power source 20, temperature controller 19 for chamber 18, and computer processor 15.

Optional computer processor 15 is connected to catalytic alloy hydrogen sensor 14 via electrical connection 13. Signal output from catalytic alloy hydrogen sensor 14 is conducted via line 13 to computer processor 15 and collected as data. Computer processor 15 is optionally connected to pressure indicator 10 via electrical connection 23 and is optionally connected to thermocouple 12 via electrical connection 5. Signal outputs from pressure indicator 10 and thermocouple 12 are optionally stored as data as well. Using software, the computer processor collects and analyzes the data and generates a control signal. The control signal is communicated via electrical connection 7 to a process control device 9. The control signal may be based on the relative or qualitative hydrogen signals from the catalytic alloy hydrogen sensor, or may be based on the mole percent hydrogen as calculated by the computer using the signal from the pressure indicator and the signal from the catalytic alloy hydrogen sensor. Optionally, a display may be used without the computer processor and the signal from the catalytic alloy hydrogen sensor or the mole percent hydrogen as manually calculated from the signal from the catalytic alloy hydrogen sensor and the signal from the pressure indicator may prompt an operator to make an adjustment to an operating parameter.

Flow from a location in a process is routed to assembly 1, typically via a conduit 22. Conduit 22 may be equipped with needle valve 24, pressure indicator 26, thermocouple 28, filter 30 and valve 32. Usually the flow in conduit 22 is a slipstream taken from a process stream or process unit. The valves 24 and 32 of conduit 22 may be configured to control the amount of process flow that is directed through assembly 1 and the amount of process flow that may be directed elsewhere such as to flare.

Optionally, a calibration conduit 34 equipped with needle valve 38 and connected to calibrations gases 36 may be used to calibrate the assembly. To calibrate the assembly, one or more gases of known amounts of hydrogen are directed at a known or measured flow rate to the conduit 3 of assembly 1 via calibration conduit 34 and valve 38 and the signal generated by catalytic alloy hydrogen sensor 14 is recorded as each of the gases pass through assembly 1. The signal generated by catalytic alloy hydrogen sensor 14 is then correlated to the known amount of hydrogen present in the gases.

The apparatus shown in FIG. 1 may be used to control a refinery or chemical process by comparing the amount of hydrogen measured using the assembly to a set of predetermined values and adjusting operating parameters as a result. Since many refinery and chemical processes use hydrogen in some way, many processes could benefit from the apparatus. Such processes include cracking, hydrocracking, aromatic alkylation, isoparaffin alkylation, isomerization, polymerization, reforming, dewaxing, hydrogenation, dehydrogenation, transalkylation, dealkylation, hydration, dehydration, hydrotreating, hydrodenitrogenation, hydrodesulfurization, methanation, ring opening, syngas shift, and hydrogen purification. A specific example is one where the assembly is used to control the processes to establish the most optimum time intervals for changing the adsorption or desorption cycles in an isomerization process which uses an adsorbent for separating hydrocarbons.

A particularly useful application of the assembly of FIG. 1 is in adsorptive separation processes where either the desorbent or the process fluid is or contains hydrogen. Monitoring the hydrogen concentration of the streams during the operation of the adsorptive separation and in regeneration of the adsorbent in conjunction with adjusting operating parameters allows for more effective control of the cycle time of the adsorber bed(s) including the timing of directing process fluid or desorbent to the beds of adsorbent. Likewise, process efficiency can be increased through the control of the flow rates of the stream to and from the adsorber beds.

One specific example of the invention involves using the apparatus in a total isomerization process (TIP). Hydrocarbon isomerization processes in general are widely used to convert normal hydrocarbons to more valuable non-normal hydrocarbons. The more valuable non-normal hydrocarbons may be used as gasoline blending components to boost the octane number of the gasoline. One class of vapor phase hydrocarbon isomerization processes uses adsorption technology to remove non-isomerized normal hydrocarbons from the isomerization reactor effluent. The adsorbed normal hydrocarbons are desorbed using hydrogen and recycled to the isomerization reactor. The overall production of the process is enhanced by keeping reactants in circulation within the process until the desired products are formed. Detailed descriptions of variations of this isomerization technique may be found in Crusher, N. A. In Handbook of Petroleum Refining Processes 2^{nd} ed.; Meyers, R. A. Ed.; McGraw-Hill: New York, 1997; pp 9.29-9.39, U.S. Pat. No. 4,210,771, U.S. Pat. No. 4,709,117, and U.S. Pat. No. 4,929,799 with the patents hereby incorporated by reference.

In the total isomerization process, a hydrocarbon-enriched stream from an isomerization zone is flowed to an adsorption zone to adsorb the normal hydrocarbons and collect the more valuable non-normal hydrocarbons. The normal hydrocarbons are desorbed from the adsorption zone using a hydrogen-enriched stream to produce the desorption effluent. The control of the streams through the adsorption zone and especially the switch from adsorption mode to desorption mode and the flow rates of the different stream are critical to the efficiency of the process. If the timing of the switch in operational modes of the adsorption-desorption cycle is not correct, valuable product may be lost or contaminated. Similarly, of the flow rates of the different stream are not periodically evaluated and if necessary adjusted, efficiency and profitability of the process decreases. There is a need for innovations to increase the precision and reliability of the control for the adsorptive separation process. Innovations that are successful can greatly improve the economics of the process.

By way of example, one embodiment of the invention as applied to the total isomerization process begins with flowing a fresh feed stream containing normal and non-normal hydrocarbons to either the isomerization reactor or the adsorption zone. A variable mass flow desorption effluent containing at least normal hydrocarbons is flowed to the isomerization reactor containing an isomerization catalyst to form a reactor effluent containing normal hydrocarbons and isomerized non-normal hydrocarbons. The reactor effluent is cooled and separated into an adsorber feed stream and a hydrogen purge gas which are each conducted to an adsorption zone containing an adsorbent capable of adsorbing the normal hydrocarbons. In the adsorption zone, the normal hydrocarbons are adsorbed and the non-normal hydrocarbons are withdrawn and collected. The normal hydrocarbons are then desorbed from the adsorption zone using the hydrogen purge gas to produce the desorption effluent.

This embodiment of the present invention provides enhanced control of the adsorption-desorption cycle and therefore increased efficiency and preservation of valuable product. The total isomerization process contains two main sections, the isomerization reactor and the adsorption zone. The fresh feed to the process is fed either to the isomerization reactor, termed the “reactor-lead” embodiment, or to the adsorption zone, termed the “adsorber-lead” embodiment. The reactor-lead embodiment is preferred when the fresh feed contains a significant amount of normal hydrocarbons, such as greater than 25 mole percent. The adsorber-lead embodiment is preferred when the fresh feed contains an appreciable amount of non-normal hydrocarbons. Reactor-lead and adsorber-lead operations are well understood in the art and are explained in detail in U.S. Pat. No. 4,929,799 which is incorporated by reference. A typical application of the total isomerization process is to isomerize normal hydrocarbons containing from about 4 to about 7 carbon atoms to form the corresponding isomeric non-normal hydrocarbons, and fresh feeds for this typical application are frequently obtained from refinery distillation operations.

The isomerization reactor, which may be one or more serially connected individual reactors, contains an isomerization catalyst that is effective for the isomerization of normal hydrocarbons to non-normal hydrocarbons. Various traditional catalysts may have insufficient activity at this low temperature, but newly developed catalysts are effective and therefore preferred. Suitable catalysts include solid strong acid catalysts where at least one member selected from Group VIII metals is supported on a support consisting of hydroxides and oxides of Group IV metals and Group III metals and mixtures thereof, with the catalyst being calcined and stabilized. Suitable catalysts are in U.S. Pat. No. 4,929,700, U.S. Pat. No. 4,709,117 and U.S. Pat. No. 4,210,771 which are incorporated herein by reference. An example of a suitable catalyst is a zeolite-type catalyst such mordenite with platinum. As hydrocarbons enter the isomerization reactor, whether from a desorption effluent (discussed below) or a combination of desorption effluent and fresh feed, normal hydrocarbons contact the catalyst and a portion of the normal hydrocarbons are isomerized to form non-normal hydrocarbons. Since the isomerization of hydrocarbons is an equilibrium-limited reaction, a portion of the normal hydrocarbon will not be isomerized and will exit the reactor in the reactor effluent. Therefore, the reactor effluent will contain at least hydrogen, normal hydrocarbons, and isomerized non-normal hydrocarbons, with the normal and non-normal hydrocarbons preferably near equilibrium proportions.

The reactor effluent is cooled and separated prior to reaching the adsorption zone using common separation techniques such as flashing in a separator drum to separate a hydrogen-enriched stream from a hydrocarbon-enriched stream. The hydrocarbon-enriched stream is used as the adsorber feed, and the hydrogen enriched stream is used as the desorbent or purge gas. The hydrogen-enriched stream contains mainly hydrogen, but if light hydrocarbons are present in the feed, the hydrogen enriched stream may also contain hydrocarbons having from one to about three carbon atoms. The hydrocarbon stream contains mainly hydrocarbons having four or more carbon atoms as well as dissolved hydrogen. Each stream is then flowed, after heat exchanging with the adsorption effluent, reactor effluent, and desorption effluent, or all three, in the vapor state to the adsorption zone. The design and operation of the adsorption zone is well known in the art and is only outlined briefly here.

The adsorber feed containing normal and non-normal hydrocarbons in the vapor state is passed at superatmospheric pressure periodically in sequence through each of a plurality of fixed adsorber beds, e.g., four as described in U.S. Pat. No. 3,700,589, hereby incorporated by reference, or three as described in U.S. Pat. No. 3,770,621, hereby incorporated by reference, of an adsorption zone with each bed containing zeolitic molecular sieve adsorbent. Preferably, the adsorbents have effective pore diameters of substantially 5 Angstroms. In a four-bed system, each of the beds cyclically undergoes the stages of:

A-1 adsorption-fill wherein the vapor in the bed void space consists principally of hydrogen purge gas with the incoming adsorber feed forcing the hydrogen purge gas from the bed void space and out of the bed without substantial intermixing of the hydrogen purge gas with the non-adsorbed adsorber feed. The term “bed void space” for purposes of this description means any space in the bed not occupied by solid material except the intracrystalline cavities of the zeolite crystals. The pores within any binder material which may be used to form agglomerates of the zeolite crystals is considered to be bed void space;

A-2 adsorption wherein the adsorber feed is cocurrently passed through the bed and the normal hydrocarbons of the adsorber feed are selectively adsorbed into the internal cavities of the crystalline zeolitic adsorbent and the nonadsorbed hydrocarbons of the adsorber feed are removed from the bed as an adsorption effluent having a greatly reduced content of non-normal hydrocarbons;

D-1 void space purging wherein the bed loaded with normal hydrocarbons to the extent that the stoichiometric point of the mass transfer zone thereof has passed between 85 and 97 percent of the length of the bed and containing in the bed void space a mixture of normal and non-normal hydrocarbons in essentially the adsorber feed proportions, is purged countercurrently, with respect to the direction of A-2 adsorption by passing a stream of hydrogen purge gas through the bed in sufficient quantity to remove the bed void space adsorber feed vapors but not more than that which produces about 50 mole percent, preferably not more than 40 mole percent, of adsorbed normal hydrocarbons in the bed effluent; and

D-2 purge desorption wherein the selectively adsorbed normal hydrocarbons are desorbed to form a desorption effluent by passing a hydrogen purge gas countercurrently with respect to A-2 adsorption through the bed until a major proportion of adsorbed normal hydrocarbons has been desorbed and the bed void space vapors consist principally of hydrogen purge gas. The hydrogen purge gas may be a hydrogen recycle stream which contains light hydrocarbons in addition to the hydrogen.

The zeolitic molecular sieve employed in the adsorption beds must be capable of selectively adsorbing the normal hydrocarbons of the adsorber feed using molecular size and configuration as the criterion. Such a molecular sieve should, therefore, have an apparent pore diameter of less than about 6 Angstroms and greater than about 4 Angstroms. A particularly suitable zeolite of this type is zeolite A, described in U.S. Pat. No. 2,883,243, which in several of its exchanged forms, notably the calcium/sodium cation form, has an apparent pore diameter of about 5 Angstroms and has a very large capacity for adsorbing normal hydrocarbons. Other suitable molecular sieves include zeolite R, U.S. Pat. No. 3,030,181, zeolite T, U.S. Pat. No. 2,950,952, and the naturally occurring, zeolitic molecular sieves chabazite and erionite. The cited U.S. patents are incorporated herein by reference.

For the adsorbents to function properly, the hydrocarbons must be maintained in the vapor state and the adsorption zone must be operated at a temperature above about 260° C. (500° F.), preferably within the range of about 260° C. (500° F.) to about 343° C. (650° F.) with the normal operating pressure of the adsorption zone being in the range of about 200 psig to about 300 psig and preferably about 250 psig. A fired heater may be installed to heat the hydrogen purge gas and the adsorber feed stream to the temperature of the adsorption zone, or heat exchange techniques may be employed.

The total isomerization process is typically controlled by a computer to monitor and set each of the various valves which control flows and flowrates to and from the adsorptive separation beds and total and partial hydrogen pressure. In the present embodiment of the invention the computer is used in conjunction with one or more catalytic alloy hydrogen sensor assemblies in order to set and control the cycle times of the adsorptive separation beds and the flow rates of associated streams. The timing of the advancement of the adsorptive beds though the stages is important in order to maximize the profitability of the overall process. If the adsorptive beds are advanced through the cycle too quickly, the full capacity of the adsorptive beds are not used and the process becomes inefficient. On the other hand, if the adsorptive beds are advanced though the stages too slowly, valuable product may be lost for the streams may be diluted with breakthrough hydrogen or normal or iso-hydrocarbons may be mixed making the separation less effective. Determination and control of the optimum timing of the advancement of the adsorptive beds though the stages results in customizing the advancement of the stages to the specific unit in operation and takes the greatest advantage of the adsorptive capacity of the beds.

Each effluent conduit from the adsorption beds may be equipped with a dedicated independent catalytic alloy hydrogen sensor assembly which are then all electrically connected to a computer, or a single catalytic alloy hydrogen sensor assembly may be in fluid communication with all of the effluent conduits. When a single assembly is used, appropriate valves would allow for sensing of only one stream at a time. Of course, any number of catalytic alloy hydrogen sensor assemblies may be employed, the scope of the invention is not limited to these two examples. Should any two effluent lines share a catalytic alloy hydrogen sensor assembly, appropriate valves would allow for sensing of one stream at a time. Although only effluents are monitored in the present embodiment, it may be beneficial to monitor feed streams or both feed and effluent streams for other applications.

Without intending any limitation on the scope of the present invention and as merely illustrative, this invention is explained below in specific terms as applied to one specific embodiment of the invention, the total isomerization of normal C₅ and C₆ hydrocarbons using a sulfated zirconia catalyst in an isomerization reactor, and a zeolitic molecular sieve adsorbent in an adsorption zone. Hydrogen is the desorbent. For ease of understanding, the process of the invention described in detail below is limited to the adsorber-lead embodiment of the invention utilizing controlled variable steam streams for additional heat exchange followed by fired heaters. Also, a great deal of processing equipment such as control valves, heat exchangers, heaters, and the like are not shown or discussed.

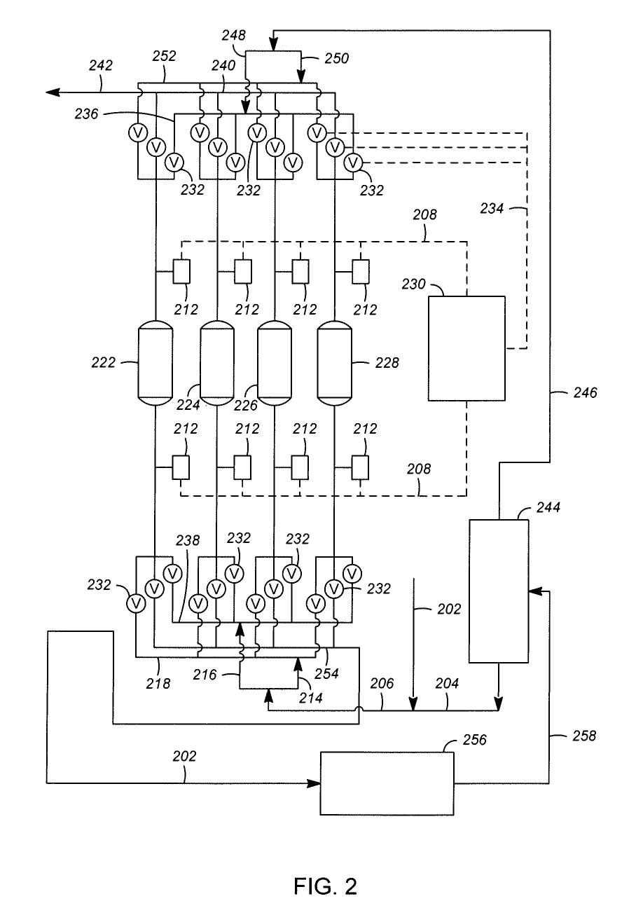

Referring now to FIG. 2, an adsorber-feed stream in line 204 and a fresh feed stream in line 202, both containing normal and non-normal C₅ and C₆ hydrocarbons, are combined to form a combined feed in line 206. A portion of the combined feed is directed into line 210 and a portion is directed into line 214. From these lines, the combined feed stream is directed to the appropriate bed in the adsorption zone. For the following description, bed 222 is undergoing A-1 adsorption-fill; bed 224, A-2 adsorption; bed 226, D-1 void space purging; and bed 228, D-2 purge desorption. A portion of the combined feed from line 206 is directed via line 214 through manifold 218 and valve 232 to adsorption bed 222 undergoing A-1 adsorption. Each of the four adsorption beds in the system, namely beds 222, 224, 226, and 228 contain a molecular sieve adsorbent in a suitable form such as cylindrical pellets.

Each of the streams to and from the adsorption beds are equipped with a catalytic alloy hydrogen sensor assembly of FIG. 1. Each of the catalytic hydrogen sensor assemblies 212 are electronically connected via lines 208 to microprocessor, such as a computer, 230. Microprocessor 230 in turn is electronically connected to the control valves 232 directing flow to and from the adsorptive separation beds via electrical connections 234. Control valves 232 also control the flow rates of the streams. For ease of understanding, FIG. 2 only shows electrical connections to three of the valves 232, when in actual practice, the connections may be to more or all of the valves 232, and other control devices as well.

Bed 222, at the time that feed passing through associated valve 232 enters, contains residual hydrogen-containing purge gas from the preceding desorption stroke. As will be explained in detail later, the hydrogen-containing purge gas is supplied to the adsorbers during desorption as a hydrogen recycle stream via. The rate of flow of the adsorber feed through line 214, manifold 218 and valve 232 is controlled such that bed 222 is flushed of residual hydrogen-containing purge gas uniformly over a period of about two minutes.

During this first stage of adsorption in bed 222, a portion of the hydrogen-containing purge gas effluent passes from the bed through the associated catalytic alloy hydrogen sensor assembly 212 where the amount of hydrogen in the bed effluent is periodically or continuously sensed and a corresponding signal is sent to computer 230. The bed effluent passes through associated valve 232 and into manifold 236. During the two minute period when the hydrogen-containing purge gas was being flushed from bed 222, the remaining combined feed passes through line 210, through manifold 238, and associated valve 232 to bed 224.

The normal paraffins in the combined feed are adsorbed by bed 224 undergoing A-2 adsorption and an adsorber effluent containing an isomerate product, i.e., the non-adsorbed non-normals, emerges from the bed and a portion of the stream passes through associated catalytic alloy hydrogen sensor assembly 212 where the amount of hydrogen in the effluent is periodically or continuously sensed. An electrical signal indicating the amount of hydrogen present sent to computer 230. The reminder of the stream is passed through associated valve 232 and manifold 240. The adsorber effluent flows through product conduit 242 where a number of operations such as cooling and separating to remove hydrogen and other low boiling materials takes place. The product non-normal hydrocarbons are collected.

During the one minute period when the residual hydrogen-containing purge gas is being flushed from bed 222, i.e., A-1 adsorption, bed 226 is undergoing the first stage of purging with the hydrogen stream wherein the hydrocarbons in the bed void space are flushed from the bed, i.e., D-1 urging. During the same two minute interval, bed 228 is undergoing the second stage of desorption, i.e., D-2 purge desorption, in which the normal hydrocarbons are desorbed from the molecular sieve adsorbent using the hydrogen stream.

From separation zone 244 the hydrogen-containing gas stream is passed through line 246 and split into two portions in lines 248 and 250. Typically, the recycle hydrogen stream has a hydrogen content from about 75% to about 95%. The recycle hydrogen stream could have a hydrogen content of up to 100%. The concentration of light hydrocarbons and other impurities are generally maintained at lower levels.

Hydrogen is passed through line 250, manifold 252, and associated valve 232 countercurrently (with respect to the previous adsorption stroke) through bed 226. The low, controlled flow rate employed for the one minute first stage desorption flushes non-adsorbed hydrocarbons from the bed voids without causing excessive desorption of the normals from the adsorbent. A portion of the effluent from bed 225 passes through associated catalytic alloy hydrogen sensor assembly 212 where the amount of hydrogen in the effluent is periodically or continuously sensed. An electrical signal indicating the amount of hydrogen present sent to computer 230. The reminder of the stream is passed through associated valve 232 and manifold 238 where it may be recycled directly to bed 224 undergoing A-2 adsorption.

The second portion of the hydrogen recycle stream in line 248 is passed through manifold 236 where it is mixed with the previously mentioned first stage adsorption effluent and then passes through associated valve 232 and bed 228. During this period, selectively adsorbed normal paraffins are desorbed from the zeolitic molecular sieve and flushed from the bed. A portion of the adsorption effluent from bed 228, comprising hydrogen and desorbed normal paraffins, is passed through associated catalytic alloy hydrogen sensor assembly 212 where the amount of hydrogen in the effluent is periodically or continuously sensed. An electrical signal indicating the amount of hydrogen present sent to computer 230. The reminder of the stream is passed through associated valve 232 and manifold 254. The effluent is recycled in line 202 to an isomerization zone 256 containing an isomerization catalyst to generate isomerization zone effluent in line 258. The isomerization zone effluent contains normal and non-normal hydrocarbons in near equilibrium proportions and hydrogen. The hydrogen is separated from the isomerization zone effluent in separation zone 224. The reminder of the isomerization zone effluent is combined with fresh feed stream 202 and introduced to the adsorptive separation zone.

The foregoing description is for a single 90-second period of a total six minute preferred cycle for the system. For the next 90-second period, appropriate valves are operated so that bed 222 begins A-2 adsorption, bed 224 begins D-1 purging, bed 226 begins D-2 desorption, and bed 228 begins A-1 adsorption. Similarly, a new cycle begins after each 90-second period and at the end of a six minute period all the beds have gone through all stages of adsorption and desorption.

The process is controlled using the catalytic alloy hydrogen sensor assemblies 212 which provide electrical information to the computer indicating the amount of hydrogen present in each of the bed effluents. The computer is also able to monitor and set each of the various valves which control flow rates. The amount of hydrogen in the bed effluents is monitored and the changing hydrogen concentrations allow for the overall process to be controlled for maximum efficiency with minimum product loss. Again, the hydrogen may be monitored qualitatively, as the hydrogen partial pressure from the hydrogen sensor alone, or quantitatively as mole percent hydrogen as calculated from the measurements of both the hydrogen sensor and the pressure indicator. Specifically, examples of operating parameters that may be adjusted as a result of monitoring the hydrogen concentration of the effluents include, the flow rates of the streams through the adsorptive separation beds and the timing of the cycling of the beds through the stages of adsorption and desorption. Different operating parameters may be adjusted for different applications. Other possible operating parameters include flow direction, pressure, temperature and different cycle times. Operating parameters may be adjusted singularly or a combination of parameters may be adjusted. The control may be done continuously or periodically. Which parameters are adjusted may be influenced by costs or profits. The control may also be applied to other operating parameters which are used in the event of the adsorbent bed issues such as deactivation or poisoning.

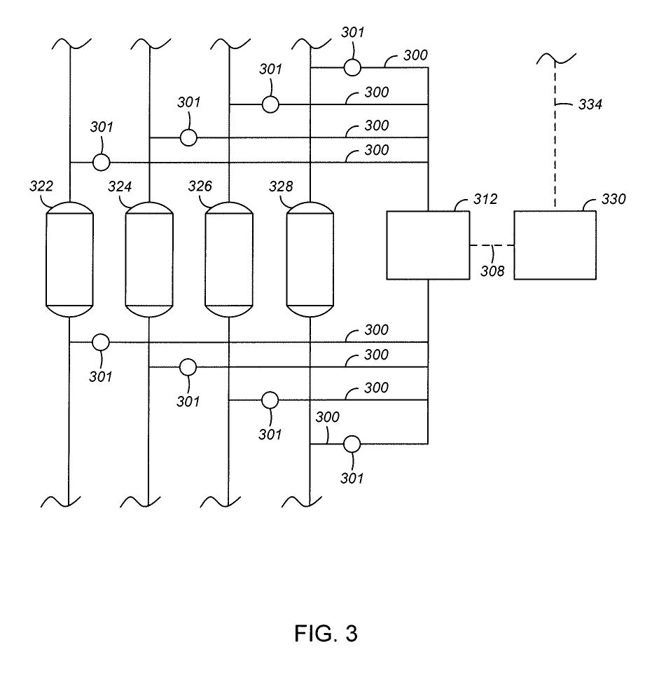

In an alternative embodiment, one catalytic alloy hydrogen sensor assembly may be used to monitor the hydrogen in a number of streams as shown in FIG. 3 which is a partial flow scheme only showing the portion of the process where the catalytic alloy hydrogen sensor assembly is found. In one embodiment, the rest of the process may be as shown in FIG. 2. Turning to FIG. 3, each adsorber bed has a slipstream 300 which is routed to a single catalytic alloy hydrogen sensor assembly 312. Which effluent is being passed through the catalytic alloy sensor assembly is controlled by the set of valves 301. The catalytic alloy hydrogen sensor assembly is electronically connected to a computer 330 by electrical connection 308. Computer 330 in turn is electronically connected via line 334 to devices to control operating parameters such as the valves controlling the streams to and from the adsorber beds. The different effluents cycle through the catalytic alloy hydrogen sensor assembly until sufficient data is collected to control operating parameters.

It must be emphasized that the above description is merely illustrative of a few embodiments and not intended as an undue limitation of the generally broad scope of the invention. Moreover, while the description is narrow in scope, one skilled in the art will understand how to extrapolate to the broader scope of the invention. For example, a reactor-lead flowscheme using the controlled variable steam streams and the heat exchangers used in conjunction with the controlled variable steam streams or a reactor-lead flow scheme using the surge drum on the desorption effluent can be readily extrapolated from the foregoing description. Furthermore, conserving the excess heat in the desorption effluent through heat exchange with only the adsorber feed or only the hydrogen purge gas, heat exchanging the adsorber feed, the hydrogen purge gas, or both, one or more times with the reactor effluent, and using a controlled variable hot oil stream in lieu of the controlled variable stream would be readily apparent to one skilled in the art.

### EXAMPLE

A total isomerization process was monitored using the present invention and adjustments were made to flow rates and cycle times of the total isomerization process based on data collected using the present invention. Previously, gas chromatograph (GC) systems were used to periodically monitor the adsorptive separation portion of the total isomerization process and adjust parameters to optimize the refining process. Therefore, a GC system was also set up to verify the data collected using the catalytic alloy hydrogen sensor. The GC system was used to monitor hydrocarbons and hydrogen at specific locations within the total isomerization process and to compare the results to data collected from the present invention. The GC system had several drawbacks. First, the GC system required a complicated manifold and care was required to watch for and correct leaks. Over time, hydrocarbons in the hydrogen stream operates to deactivate the GC system and the sampling frequency is limited to every ten seconds. The GC columns need to be handled and shipped with care and the columns are susceptible to plugging.

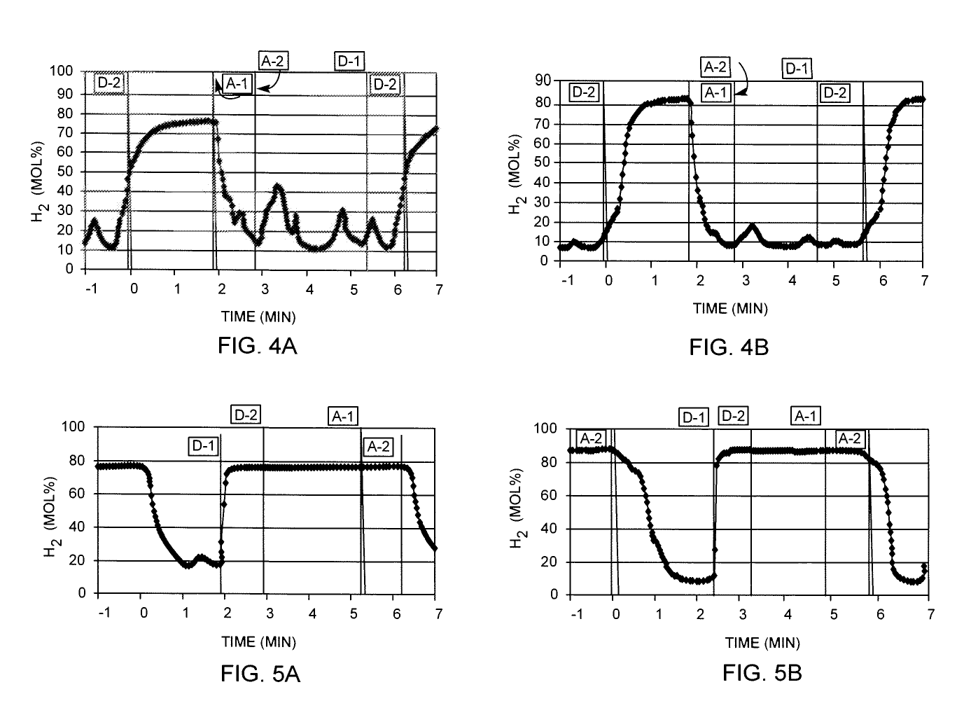

FIG. 4A and 4B show plots of the data collected by the present invention sampled at the bottom of a selected adsorber before and after operating parameter adjustments were made. Time is presented along the x-axis and the concentration of hydrogen is provided along the y-axis. The point at which the bed was cycled to a different stage is noted. The hydrogen concentration of the effluent was monitored continuously for at least one complete cycle and preferably several cycles. When the flow is through the adsorber so that the effluent is at the bottom, it is the D-1 to D-2 cycle change that is monitored. Hydrogen is expected to increase in the effluent when the adsorber undergoes D-2 desorption as the adsorbed components are desorbed and carried with the hydrogen desorbent. When most of the adsorbed components have been desorbed, the hydrogen content of the effluent will approach a constant amount indicating that the D-2 desorption is complete. When the adsorber is undergoing A-1 adsorption, residual hydrogen is quickly purged out of the adsorber as shown by the rapid decrease of hydrogen in the effluent, thus the rapid drop in hydrogen detected by the current invention.

In FIG. 4A which is the plot of the data before any adjustments were made, the hydrogen concentration is seen to fluctuate during the A-2 and D-1 stage. This is an indication of inadequate tuning of the cycle as breakthrough hydrogen is being detected. Also, the cycle to the D-2 stage is occurring after the hydrogen in the effluent has reached a significant level, approximately 50 mole-%. Based upon this data adjustments were made to the operating parameters of the adsorptive separation portion of the total isomerization process. For example, the overall cycle time was shortened and the timing for the D-2 step was changed. Also adjustments were made to the D-1 purge. The amount of hydrogen in the effluent was again monitored after the adjustments were made. The data collected after the adjustments is plotted in FIG. 4B. In comparing FIGS. 4A and 4B, it is readily apparent that the hydrogen fluctuation during the A-1 stage is dramatically reduced. Also, the advancement of the cycle to the D-2 stage occurs before the hydrogen in the effluent reaches an appreciable amount, approximately 15 mole %.

FIGS. 5A and SB show plots of the data collected by the present invention installed at the top of a selected adsorber before and after operating parameter adjustments were made. Again, time is presented along the x-axis and the concentration of hydrogen is provided along the y-axis. The point at which the bed was cycled to a different stage is noted. The hydrogen concentration of the effluent was monitored continuously for at least one complete cycle and preferably several cycles. When the flow is through the adsorber so that the effluent is at the top, it is the A-1 to A-2 cycle step that is monitored. Hydrogen is expected to remain high during adsorption purge stage A-1 while hydrogen is being forced through the adsorber by the incoming hydrocarbon stream. When the isomerate product begins to elute the concentration of hydrogen in the effluent will begin to decrease. The cycle should be advanced to A-2 at the point when the hydrogen concentration begins to drop to collect the greatest amount of isomerized product and prevent the recycle of product back to the reactors. When the amount of hydrogen approaches a lower limit indicating the adsorption capacity of the adsorber has been reached, the cycle is stepped so that the adsorber begins to undergo D-2 desorption where the lesser adsorbed components are desorbed and carried with the hydrogen desorbent. The result is less contamination of the desired product as hydrogen and normal paraffins are not being mixed with the adsorber feed.

In FIG. 5A which is the plot of the data before any adjustments were made, the hydrogen concentration is seen to fluctuate during the A-2 stage. This is an indication of un-optimized conditions. The A-2 step starts with high hydrogen content. The hydrogen contains residual normal alkanes that contaminate the product. Based upon this data adjustments were made to the operating parameters of the adsorptive separation portion of the total isomerization process. For example, the A-1 step was adjusted to a shorter time and the A-1 flow rate was increased. The amount of hydrogen in the effluent was again monitored after the adjustments were made. The data collected after the adjustments is plotted in FIG. 5B. In comparing FIGS. 5A and 5B, it is readily apparent that the hydrogen fluctuation during the A-2 stage is dramatically reduced. The transition to the A-1 step occurs as the hydrogen concentration falls. Less hydrogen is used in A-2 thereby providing more efficient use of hydrogen for the D-2 step and less contamination of the product with low octane normal alkanes.

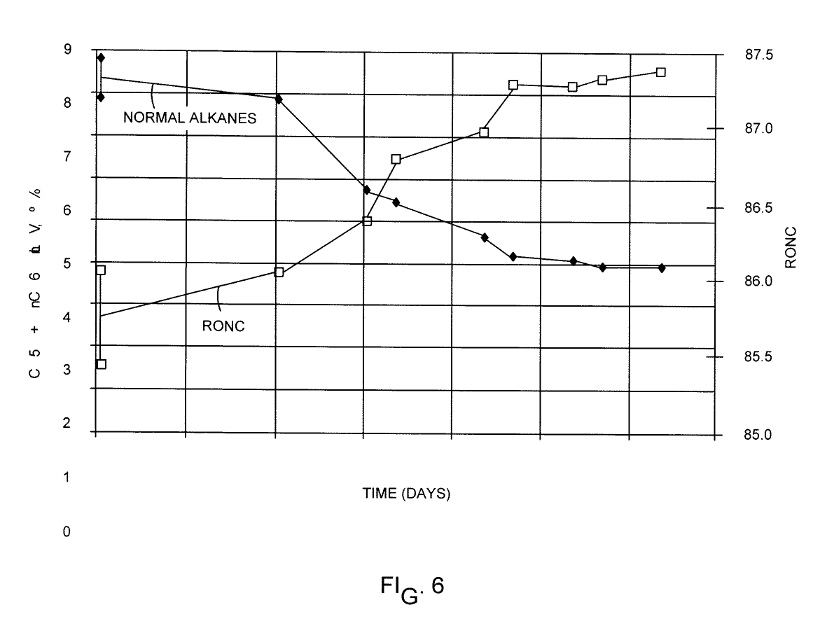

The overall product from the total isomerization process was monitored over time for the content of normal alkanes and the research octane number in order to measure the effectiveness of the control of the process using the invention. FIG. 6 shows a plot of the data over a seven day period. Time in days is shown on the x-axis, the left hand y-axis shows the liquid volume percent of pentane and hexane, and the right hand y-axis show the research octane number of the product. As can been seen from the plot over the seven day period there was a decline in the amount of normal alkanes in the product while at the same time there was an increase in the research octane number of the process. Since a goal of the total isomerization process is to produce the highly valued higher octane isomerized components, data of FIG. 6 clearly shows that the invention measurably improved the product of the total isomerization process.

What is claimed is:

1) A process for sensing hydrogen in a refinery process stream or a chemical process stream comprising:
(a) flowing at least a portion of the refinery process stream or the chemical process stream through a conduit which is part of a main support and through a series of flow components which are attached to the main support and in fluid communication with the conduit wherein the flow components comprise:
(i) needle valve
(ii) pressure indicator
(iii) catalytic alloy hydrogen sensor, and
(iv) back pressure regulator
(b) generating a signal indicating the amount of hydrogen in the stream using the catalytic alloy hydrogen sensor and communicating the signal to a computer processor.

2) The process of claim 1 further comprising additional flow components of a filter, a check valve, and a thermocouple.

3) The process of claim 2 wherein the portion of the refinery process stream or the chemical process stream is flowed through the flow components in the order of the needle valve, the filter, the check valve, the pressure indicator, the thermocouple, the catalytic alloy hydrogen sensor and the back pressure regulator.

4) The process of claim 1 further comprising controlling the temperature of the catalytic alloy hydrogen sensor.

5) The process of claim 4 wherein the temperature of the catalytic alloy hydrogen sensor is controlled using an integrated temperature control.

6) The process of claim 1 further comprising monitoring the amount of hydrogen in the stream over time.

7) The process of claim 1 further comprising generating a signal indicating the pressure in the stream using the pressure indicator and communicating the signal to a computer processor.

8) The process of claim 6 further comprising calculating, in the computer processor, a quantitative amount of hydrogen in the stream from the signal indicating the pressure from the pressure indicator and the signal indicating the amount of hydrogen from the catalytic alloy hydrogen sensor.

9) The process of claim 8 wherein the quantitative amount of hydrogen is selected from the group consisting of mole percent of hydrogen, mass percent of hydrogen, and volume percent of hydrogen.

10) The process of claim 1 wherein the catalytic alloy hydrogen sensor is a palladium-nickel catalytic alloy hydrogen sensor.

11) The process of claim 1 further comprising, adjusting an operating parameter based upon the signal indicating the amount of hydrogen in the stream.

12) The process of claim 1 further comprising, adjusting an operating parameter based upon a set of signals indicating the amount of hydrogen in the stream collected over time indicating the amount of hydrogen in the stream.

13) The process of claim 3 wherein the operating parameter is selected from the group consisting of, flow direction, flow rate, pressure, temperature, and cycle time.

14) The process of claim 1 further comprising controlling the environment within a chamber housing the main support and flow components.

15) The process of claim 1 further comprising controlling the temperature within a chamber housing the main support and flow components.

16) The process of claim 1 further comprising providing power to at least one flow component.

17) The process of claim 1 further comprising calibrating the catalytic alloy hydrogen sensor prior to step (a) of claim 1.

18) The process of claim 1 wherein the temperature, pressure, and flow rate of the portion of the process stream is monitored and the portion of the process stream is filtered before entering the conduit.

19) The process of claim 1 wherein the remainder of the refinery process stream or chemical process stream is the feed stream to or effluent of an adsorber bed in an adsorptive separation process.

20) The process of claim 19 wherein the refinery process stream is selected from the group consisting of an effluent of an isomerization zone being conducted to an adsorptive separation zone; the effluent of an adsorptive separation zone being recycled to an isomerization zone, the effluent of an adsorptive separation zone being recycled as desorbent; the effluent of an adsorptive separation zone being collected as product.

21) The process of claim 1 wherein the signal indicating the amount of hydrogen in the stream is generated continuously or periodically.

22) A process for controlling a refinery or chemical process comprising:
(a) flowing at least a portion of a refinery or chemical process stream through a flow conduit in a main support with flow components attached to the main support and interacting with the flow conduit, said flow components comprising
(i) a needle valve
(ii) a pressure indicator
(iii) a catalytic alloy hydrogen sensor, and
(iv) an back pressure regulator
(b) generating a signal indicating the amount of hydrogen in the stream using the catalytic alloy hydrogen sensor and communicating the signal to a computer processor; and
(c) adjusting an operating parameter of the refinery or chemical process based upon the signal indicating the amount of hydrogen in the stream.

23) The process for controlling of claim 22 further comprising additional flow components of a filter, a check valve, and a thermocouple.

24) The process for controlling of claim 23 wherein the portion of the refinery process stream or the chemical process stream is flowed through the flow components in the order of the needle valve, the filter, the check valve, the pressure indicator, the thermocouple, the catalytic alloy hydrogen sensor and the back pressure regulator.

25) The process for controlling of claim 22 further comprising controlling the temperature of the catalytic alloy hydrogen sensor.

26) The process for controlling of claim 22 further comprising monitoring the amount of hydrogen in the stream over time.

27) The process for controlling of claim 22 wherein the catalytic alloy hydrogen sensor is a palladium-nickel catalytic alloy hydrogen sensor.

28) The process for controlling of claim 22 further comprising:
(a) flowing at least a portion of additional refinery or chemical process streams through additional flow conduits in additional main supports having the flow components;
(b) generating additional signals indicating the amount of hydrogen in the streams using the catalytic alloy hydrogen sensors and communicating the additional signals to a computer processor; and
(c) adjusting additional operating parameters of the refinery or chemical process based upon the signals indicating the amount of hydrogen in the streams.

29) The process for controlling of claim 22 wherein:
(a) the refinery or chemical process is an adsorptive separation process;
(b) the process stream is the effluent from or the feed to an adsorber bed containing adsorbent; and
(c) the operating parameter is selected from the group consisting of the cycle time of the adsorber beds and the flow rate of the fluid through the adsorber beds.

30) The process for controlling of claim 22 wherein:
(a) the refinery process is a total isomerization process;
(b) the process stream is the effluent from or the feed to an adsorber bed containing adsorbent in an adsorptive separation zone of the total isomerization process; and
(c) the operating parameter is selected from the group consisting of the cycle time of the adsorber beds and the flow rate of the fluid through the adsorber beds.

31) A process for controlling a refinery or chemical process comprising:
(a) flowing at least a portion of a refinery or chemical process stream through a flow conduit in a main support with flow components attached to the main support and interacting with the flow conduit, said flow components comprising
(i) a needle valve
(ii) a pressure indicator
(iii) a catalytic alloy hydrogen sensor, and
(iv) a back pressure regulator
(b) generating a signal indicating the amount of hydrogen in the stream using the catalytic alloy hydrogen sensor and communicating the signal to a computer processor;
(c) generating a signal indicating the pressure in the stream using the pressure indicator and communicating the signal to a computer processor;
(d) calculating, in the computer processor, a quantitative amount of hydrogen in the stream from the signal indicating the pressure from the pressure indicator and the signal indicating the amount of hydrogen from the catalytic alloy hydrogen sensor, and
(e) adjusting an operating parameter of the refinery or chemical process based upon the mole percent of hydrogen in the stream.

32) The process for controlling of claim 31 wherein the quantitative amount of hydrogen is selected from the group consisting of mole percent of hydrogen, mass percent of hydrogen, and volume percent of hydrogen.

33) The process for controlling of claim 31 further comprising additional flow components of a filter, a check valve, and a thermocouple.

34) The process for controlling of claim 33 wherein the portion of the refinery process stream or the chemical process stream is flowed through the flow components in the order of the needle valve, the filter, the check valve, the pressure indicator, the thermocouple, the catalytic alloy hydrogen sensor and the back pressure regulator.
